(19) United States
(12) Patent Application Publication
(10) Pub. No.: US 2015/0299790 A1
(43) Pub. Date: Oct. 22, 2015

(54) NOVEL ASSAY FOR MONITORING GLUCOSE BALANCE AND OXIDATIVE STRESS

(71) Applicant: THE MEDICAL RESEARCH INFRASTRUCTURE AND HEALTH SERVICES FUND OF THE TEL AVIV MEDICAL CENTER, Tel Aviv (IL)

(72) Inventor: Nachum Vaisman, Kfar Mordechai (IL)

(21) Appl. No.: 14/405,854

(22) PCT Filed: Jun. 4, 2013
(86) PCT No.: PCT/IL2013/050479

§ 371 Date: Dec. 5, 2014

Related U.S. Application Data
(60) Provisional application No. 61/655,523, filed on Jun. 5, 2012.

(51) Int. Cl. C12Q 1/68

(52) U.S. Cl. CPC C12Q 1/6883; C12Q 2600/158; C12Q 2600/106

(57) ABSTRACT

The invention relates to diagnostic and prognostic assays, particularly to methods of determining the metabolic state of a subject. More specifically, the invention provides assays and methods comprising determining the level of m RNA of uncoupling protein 2 (UCP2) specifically in platelets of the subject. The invention is useful in determining or adjusting the treatment of conditions associated with an unbalanced metabolic state manifested by elevated blood levels of glucose, reactive oxygen species (ROS) and/or free fatty acids (FFA). In particular embodiments, the assays and methods of the invention are useful in prognosing and monitoring subjects diagnosed with diabetes, particularly type II diabetes.

### FIELD OF THE INVENTION

The invention relates to diagnostic and prognostic assays, particularly to methods of determining a metabolic state of a subject based on levels of mRNA of uncoupling proteins in platelets.

### BACKGROUND OF THE INVENTION

Diabetes mellitus is on the rise worldwide and is considered to be at an epidemic level by the World Health Organization. The clinical manifestation and progression of diabetes often vary considerably between countries and commonly between ethnic groups in the same country. Currently diabetes affects 151 million people worldwide and an estimate 300 million people in 2025. There are two main forms of diabetes. Type 1 (insulin-dependent diabetes mellitus, IDDM) is due primarily to autoimmune-mediated destruction of pancreatic β-cells, resulting in absolute insulin deficiency. It is the second most common chronic disease of children. By contrast, type 2 diabetes (T2DM or T2D; non-insulin-dependent diabetes mellitus, NIDDM) is characterized by insulin-resistance and inadequate insulin secretion. The disease course is also accompanied by different metabolic responses, including increased utilization of fat deposits causing an increase in free fatty acids in the blood, and formation of free radicals and increased oxidative stress.

Unfortunately, currently there are no cures for diabetes. Treatment involves control of hyperglycemia to relieve symptoms and prevent complications while minimizing hypoglycemic episodes. Thus, the first therapeutic goal is to achieve a state of glycemic control early upon diagnosis. Accordingly, assays for evaluating the glycemic control status are necessary for adjusting the course of treatment of the patients and for monitoring their status along the disease course.

Glycemic control is currently evaluated by glycosylated hemoglobin (Hemoglobin A1c or HgbA1c) which reflects glucose control over the preceding 2 to 3 months, or by assaying glucose levels daily during fasting or two hours post-prandial, and is typically defined as blood glucose under 180 mg/dL two hours post prandial and HbA_{1c} levels <7%. Glycemic control may also be characterized by blood glucose between 80 and 120 mg/dL (4.4 and 6.7 mmol/L) during the day and between 100 and 140 mg/dL (5.6 and 7.8 mmol/L) at bedtime.

HbA1c values reflect the long-going (3 months) glycemic control status of the patients and may sometimes differ from that suggested by daily glucose readings. False elevations may occur with renal insufficiency (urea interferes with the assay), low RBC turnover (as occurs with iron, folate, or vitamin B12 deficiency anemia), high-dose aspirin, and high blood alcohol concentrations. Falsely normal values occur with increased RBC turnover, as occurs with hemolytic anemias and hemoglobinopathies (e.g., HbS, HbC) or during treatment of deficiency anemias.

Uncoupling proteins (UCPs) are homologous proteins belonging to a subfamily of mitochondrial anion carriers. UCPs uncouple oxidative metabolism from ATP synthesis and dissipate energy through heat, and thus play an important role in thermogenesis. By now, five proteins have been identified and they reside in different tissues.

UCP1 was found in brown adipose tissue, and has been reported to play an important role for energy homeostasis in rodents and neonates of larger mammals, including humans, by increasing heat production in low-temperature situations. It was also found to be induced by overfeeding.

UCP2 and UCP3 are the products of adjacent genes located on human chromosome 11 at loci genetically linked to obesity and diabetes, and both are expressed in adult humans. UCP2 mRNA is widely expressed and it is at high levels in white adipose tissue, spleen, lung, intestine and pancreatic beta cells, while UCP2 protein has been detected in fewer tissues. UCP3 mRNA is strongly expressed in skeletal muscle and to a lesser extent in heart and white and brown adipose tissues. UCP4 is present in brain and the mRNA for UCP5 is present in brain and liver. The homology of both UCP2 and UCP3 (55% and 57%, respectively) with UCP1 has led to the assumption that each of them has a specific proton/anion carrier function. The current hypothesis proposes several physiological functions for UCP2 and UCP3 such as: 1) attenuation of reactive oxygen species production and protection against oxidative stress which play important roles in minimizing ROS emission from the electron transport chain; 2) involvement in fatty acid handling and/or transport; 3) a signaling role in pancreatic beta cells, where it attenuates glucose-induced insulin secretion; and 4) thermogenic function especially for UCP3.

A series of studies showed polymorphisms of UCP genes associated with fat metabolism, obesity and diabetes. In a number of genetic studies, the relationship between UCP2 locus and susceptibility to T2DM or obesity has been suggested. For example, the −866G/A polymorphism was found to contribute to the biological variation of insulin secretion and consequently to the susceptibility to T2DM. (Marmontel et al., 2011; Emre et al., 2010; Mailloux et al., 2011; Jia et al., 2009; Azzu et al., 2010).

US2002127600 relates to human UCP2, compositions thereof and methods of using same. Disclosed is a method for diagnosing body weight disorders, said method comprising detecting in a patient sample the level of a UCP2 polypeptide or mRNA having specified sequences.

US2003036646 relates to novel human uncoupling polypeptides and isolated nucleic acids containing the coding regions of the genes encoding such polypeptides, compositions thereof and methods of using same. Also disclosed are methods of diagnosing a pathological condition or a susceptibility to a pathological condition in a subject comprising: determining the presence or absence of a mutation in the disclosed polynucleotide, or determining the presence or amount of expression of the disclosed polypeptide in a biological sample; and diagnosing a pathological condition or a susceptibility to a pathological condition based on the presence or absence of said mutation, or on the presence or amount of expression of the polypeptide.

US2011263447 relates to an in vitro method for diagnosis or risk assessment of T2D in a critical person, said method comprising determining the expression level of at least two T2D marker genes, selected from a list of over 50 markers including inter alia UCP2, in a biological sample taken from said person; and utilizing the profile of the expression levels of said T2D genes to diagnose the susceptibility of said person for T2D.

None of the art discloses or suggests evaluating the short term glycemic control status of a diabetic patient as well as their oxidative stress status using a single marker. No diagnostic assays for metabolic diseases that are based on determining the presence of analytes in isolated platelets have been approved for clinical use. There remains an unmet medical need for assays evaluating the metabolic state of patients afflicted with diabetes, or other conditions in which the outcome or prognosis is associated with balanced blood levels of glucose, reactive oxygen species (ROS) and/or free fatty acids (FFA).

### SUMMARY OF THE INVENTION

The invention relates to diagnostic and prognostic assays, particularly to methods of determining the metabolic state of a subject. More specifically, the invention provides assays and methods comprising determining the level of mRNA of uncoupling protein 2 (UCP2) specifically in platelets of the subject. The invention is useful in determining or adjusting the treatment of conditions associated with an unbalanced metabolic state manifested by abnormal blood levels of glucose, reactive oxygen species (ROS) and/or free fatty acids (FFA). In particular embodiments, the assays and methods of the invention are useful in prognosing and monitoring subjects diagnosed with diabetes, particularly type II diabetes.

The present invention is based, in part, on the surprising discovery that the level of UCP2 mRNA is elevated specifically in platelets of non-balanced diabetic patients and decreases after treatment, to a level comparable to that of healthy subjects. In contrast, the protein level of UCP2 did not differ in platelets isolated from balanced or non-balanced diabetic patients, or from healthy subjects.

The invention is further based, in part, on the unexpected discovery that platelet UCP2 mRNA correlated with the metabolic state of diabetic subjects throughout the course of treatment; a reduction of UCP2 mRNA could be detected as early as two weeks from the onset of treatment, whereas a reduction in glycosylated hemoglobin (Hemoglobin A1c or HgbA1c) could only be detected several months from the onset of treatment. Thus, platelet UCP2 mRNA is herein demonstrated to be a sensitive marker that is advantageous in treatment monitoring and early adjustment of therapeutic modalities and dosing regimens to individual patients.

The invention relates in certain aspects to methods and assays for determining the glycemic control of a subject, e.g. a diabetic subject. The invention relates in other aspects to methods and assays for determining the metabolic status of a subject, e.g. a subject with a chronic or metabolic disorder in which the outcome or prognosis is associated with balanced blood levels of glucose, reactive oxygen species (ROS) and/or free fatty acids (FFA). The invention relates in yet further aspects to methods and assays for monitoring, prognosing or determining the suitability of a treatment to a subject, e.g. a diabetic subject or a subject afflicted with a chronic or metabolic disorder as described above.

The methods and assays of the invention comprise determining (or means for determining) the level of mRNA of uncoupling protein 2 (UCP2) specifically in platelets of said subject.

Thus, according to a first aspect of the present invention, there is provided a method for determining the glycemic control status of a subject, comprising determining the level of UCP2 mRNA specifically in platelets of said subject. According to some embodiments the platelets are at least partially purified or isolated from other blood components. In another embodiment the platelets are isolated from a blood sample obtained from the subject.

According to embodiments of the invention, a platelet UCP2 mRNA level that is significantly higher than its level in healthy control subjects indicates an unfavorable glycemic control status in said subject. An unfavorable or unbalanced glycemic control status is a pathological disease state typically associated with blood glucose that are over 180 mg/dL two hours post prandial and/or HbA1c levels >7%, as detailed below. In some embodiments, diabetic patients achieving glycemic control (a favorable glycemic control status) may also characterized by a balanced oxidative status and restoration of normal ROS and/or FFA levels, as detailed below.

As the life span of platelets is ten days and they do not have a nucleus, the mRNA in freshly isolated platelets reflects, at most, the status 10-14 days prior to the date of measurement (corresponding to sample isolation); typically the mRNA in platelets will reflect the metabolic status at 5-7 days prior to the measurement. Thus, according to some embodiments, the methods and assays of the invention are used for determining the glycemic control status of said subject within the preceding 5-14 days. In other embodiments, said methods and assays are used for determining the glycemic control status of said subject within the preceding 5-7 days prior to platelet isolation.

In another embodiment, the subject is afflicted with diabetes mellitus. In a particular embodiment, said subject is afflicted with type II diabetes mellitus.

In another embodiment the method further comprises assessing the metabolic status of the subject, wherein a platelet UCP2 mRNA level that is significantly higher than its level in healthy control subjects indicates an unfavorable metabolic status in said subject.

In another aspect, the invention provides a method for determining the metabolic status of a subject, comprising determining the level of UCP2 mRNA specifically in platelets of said subject. According to some embodiments the platelets are isolated from other blood components. In another embodiment the platelets are isolated from a blood sample obtained from the subject.

In another embodiment a platelet UCP2 mRNA level that is significantly higher than its level in healthy control subjects indicates an unfavorable metabolic status in said subject.

In some embodiments, the subject is afflicted with a chronic disorder associated with abnormal or elevated blood levels of glucose, ROS or FFA. In other embodiments, the disorder is selected from the group consisting of diabetes mellitus, a nutritional disorder, a chronic inflammatory disease and an endocrine disease. For example the disorder may be selected from the group consisting of type II diabetes, type I diabetes, anorexia nervosa, obesity, syndrome X, sepsis, inflammatory bowel disease (IBD), rheumatoid arthritis (RA), chronic obstructive pulmonary disease (COPD), Hashimoto's thyroiditis and Graves' disease. In a particular embodiment said disorder is type II diabetes mellitus.

In another embodiment said disorder is type II diabetes mellitus and the method further comprises determining the glycemic control status of a subject, wherein a platelet UCP2 mRNA level that is significantly higher than its level in healthy control subjects indicates an unfavorable glycemic control status in said subject

For example, the methods and assays of the invention may be performed by using a method comprising:
a) obtaining platelets from the subject (e.g. from a blood sample of said subject);
b) determining the level of UCP2 mRNA in the platelets;
c) comparing the level of UCP2 mRNA to a control platelet UCP2 mRNA level (e.g. the level in platelets of healthy control subject(s), in a panel of control samples from a set of individuals, in a stored set of data from control individuals, or in a sample previously obtained from the same subject during a balanced state of the disease);
   wherein a level of UCP2 mRNA that is significantly (statistically significant or significant to one of skill in the art) elevated compared to the control level indicates that the subject has an unfavorable or unbalanced glycemic control or metabolic state.

The methods of the invention may further comprise the step of evaluating or selecting a therapy for said subject. For example, a level of platelet UCP2 mRNA that is significantly elevated compared to the control level (e.g. after 5-7 days or in other embodiments 5-14 days of the onset of a treatment) indicates that the treatment is unsuitable for said subject.

In another aspect there is provided a method for evaluating a treatment to a disorder in a subject in need thereof, wherein the subject is receiving the treatment for the disorder, and wherein a platelet level of UCP2 mRNA that is significantly elevated compared to its level in healthy control subjects indicates that said treatment is unsuitable for said subject.

In one embodiment the method is used for evaluating said treatment within 2-16 weeks from the onset of said treatment. In another embodiment the subject is afflicted with a chronic disorder associated with elevated or abnormal blood levels of glucose, ROS or FFA selected from the group consisting of diabetes mellitus, a nutritional disorder, a chronic inflammatory disease and an endocrine disease. In another embodiment the disorder is selected from the group consisting of type II diabetes, type I diabetes, anorexia nervosa, obesity, syndrome X, sepsis, inflammatory bowel disease (IBD), rheumatoid arthritis (RA), chronic obstructive pulmonary disease (COPD), Hashimoto's thyroiditis and Graves' disease. In a particular embodiment said disorder is type II diabetes mellitus.

Determining, measuring or quantifying the level of UCP2 mRNA and UCP2 protein may be performed by a variety of methods well known in the art. For example, the methods and assays of the invention may include, without limitation, the use of: RT-PCR, real-time RT-PCR, Oligonucleotide microarray or Northern blot.

In another aspect, there is provided a kit (or assay) comprising means for determining the level of UCP2 mRNA, and at least one of: i) means for isolating platelets from a sample and ii) instructions for determining the level of UCP2 mRNA in isolated platelets. The kit may optionally further comprise a control sample or data useful for comparing the level of UCP2 mRNA to its level in healthy control subject(s).

In one embodiment the kit comprises instructions for determining the level of UCP2 mRNA in platelets isolated from a patient afflicted with type II diabetes, type I diabetes, anorexia nervosa, obesity, syndrome X, sepsis, inflammatory bowel disease (IBD), rheumatoid arthritis (RA), chronic obstructive pulmonary disease (COPD), Hashimoto's thyroiditis or Graves' disease. In another embodiment the instructions indicate that a platelet UCP2 mRNA level that is significantly higher than its level in healthy control subjects indicates an unfavorable metabolic status in said subject. In another embodiment the instructions indicate that a platelet UCP2 mRNA level that is significantly higher than its level in healthy control subjects indicates an unfavorable glycemic control status in said subject.

In another embodiment determining the level of UCP2 mRNA is performed by a method selected from the group consisting of reverse-transcriptase polymerase chain reaction (RT-PCR), real-time RT-PCR, Oligonucleotide microarray and Northern blot.

Other objects, features and advantages of the present invention will become clear from the following description and drawings.

### BRIEF DESCRIPTION OF THE DRAWINGS

FIG. 1 depicts UCP2 mRNA levels in platelets of healthy subjects, non-balanced diabetic subjects and balanced diabetic subjects.

FIG. 2 demonstrates UCP2 mRNA levels in platelets of diabetic subjects before and after achieving glycemic control

FIG. 3 depicts UCP2 protein levels in platelets of healthy subjects, non-balanced diabetic subjects and balanced diabetic subjects.

### DETAILED DESCRIPTION OF PREFERRED EMBODIMENTS

The invention relates to diagnostic and prognostic assays, particularly to methods of determining the metabolic state of a subject. More specifically, the invention provides assays and methods comprising determining the level of mRNA of uncoupling protein 2 (UCP2) specifically in platelets of the subject. The invention is useful in determining or adjusting the treatment course of conditions associated with an unbalanced metabolic state manifested by elevated blood levels of glucose, reactive oxygen species (ROS) and/or free fatty acids (FFA). In particular embodiments, the assays and methods of the invention are useful in prognosing and monitoring subjects diagnosed with diabetes, particularly type II diabetes.

There remains an unmet medical need for laboratory tests, monitoring in real time the short term status of glucose control and oxidative stress of diabetes and other chronic metabolic diseases, as well as the acute state of these diseases during relapse. Glucose control is currently studied either by glycosylated hemoglobin (Hemoglobin A1c or HgbA1c) which reflects glucose control over the last three months, or by assaying glucose levels daily during fasting or two hours post-prandial. Oxidative stress is not monitored routinely. The inflammatory state which may be associated with oxidative stress is currently monitored by erythrocyte sedimentation rate (ESR) or C-reactive protein (CRP).

The invention unexpectedly provides a minimally invasive, easy to perform, repeatable and reliable test for monitoring the changes in glucose control and oxidative stress in response to a given treatment. The invention provides for the first time a single diagnostic assay for assessing glucose control and metabolic balance during a shortened period of several days, without requiring multiple tests or repeated/daily sampling.

The invention relates in certain aspects to methods and assays for determining the glycemic control of a subject, e.g. a diabetic subject. The invention relates in other aspects to methods and assays for determining the metabolic status of a subject, e.g. a subject with a chronic or metabolic disorder in which the outcome or prognosis is associated with balanced blood levels of glucose, reactive oxygen species (ROS) and/or free fatty acids (FFA). The invention relates in yet further aspects to methods and assays for monitoring, prognosing or determining the suitability of a treatment to a subject, e.g. a diabetic subject or a subject afflicted with a chronic or metabolic disorder as described above.

Further particular embodiments in accordance with the principles of the invention are detailed herein.

Metabolic State and Glycemic Control

According to embodiments of the invention, the methods of the invention comprise determining the level of UCP2 mRNA in isolated platelets of said subject. In one embodiment, a UCP2 mRNA level that is significantly higher than its level in isolated platelets of healthy control subjects indicates an unfavorable metabolic status in said subject. In another embodiment, a UCP2 mRNA level that is significantly higher than its level in isolated platelets of healthy control subjects indicates an unfavorable glycemic control status in said subject.

In some embodiments, the invention provides for methods and means for determining the metabolic status of a subject. In other words, the methods and kits of the invention may be used for determining whether the metabolic status of a subject is balanced (favorable) or unbalanced (unfavorable).

A favorable metabolic status is defined as a balanced energy homeostasis, characterized by blood levels of glucose, ROS and FFA that are equivalent to those of healthy subjects (within the range of average levels for the healthy population). Accordingly, an unfavorable metabolic status as used herein refers to blood levels of glucose, ROS and/or FFA that are abnormal, i.e. significantly altered compared to their respective levels in healthy control subjects (e.g. as evaluated by a physician or skilled artisan). The term unfavorable metabolic refers in some embodiments to blood levels of glucose, ROS and/or FFA that are significantly enhanced compared to their respective levels in healthy control subjects (e.g. as evaluated by a physician or skilled artisan). An unfavorable metabolic status may result from abnormal metabolism which may involve glucose (carbohydrate) and/or fatty acid oxidation pathways. When aberrations in fatty acid oxidation pathways are involved, the unfavorable metabolic status is typically manifested by ROS blood levels that are significantly enhanced compared to healthy control subjects and/or by abnormal FFA blood levels. These aberrations may also be manifested by elevated blood levels of oxidized low density lipoproteins (LDL). When aberrations in glucose metabolism are involved, glucose blood levels are typically significantly enhanced compared to healthy control subjects. As used herein, a patient with significantly enhanced blood glucose levels that do not exceed the threshold for unbalanced glycemic control (as defined below) will be defined as having an unfavorable metabolic status if said enhancement is accompanied by abnormal blood ROS and/or FFA values, as described herein.

An unbalanced metabolic state may also be evaluated by said physician or skilled artisan by considering the energy intake and various energy consumption and utilization parameters, as known in the art. For example, without limitation, parameters at the cellular level such as cellular (e.g. platelet) ATP production and cellular oxidation, and parameters at the whole body level such as respiratory quotient (RQ) may be evaluated to determine the metabolic status of the subject. For example, by comparing the relative ratio of such parameters between healthy and sick patients the skilled artisan may evaluate the metabolic status of the subject compared to healthy controls. An unfavorable metabolic status may be found in patients afflicted with chronic metabolic and/or inflammatory disorders that are not adequately treated or balanced by a suitable therapeutic regimen, as detailed below.

The term “metabolic disease” or “metabolic disorder” refers to a group of identified disorders in which errors of metabolism, imbalances in metabolism, or sub-optimal metabolism occur, which may involve glucose (carbohydrate), fatty acid and/or protein oxidation pathways. Accordingly, when unbalanced, these disorders are typically manifested by an unfavorable metabolic status characterized by abnormal blood levels of glucose, ROS and/or FFA compared to their respective levels in healthy control subjects, as described herein. Such disorders include without limitation diabetes and disorders associated with nutritional or endocrine imbalance.

An unfavorable metabolic status may also occur as a result of chronic inflammatory disorders, in which a non-resolving, unbalanced inflammatory process is accompanied by secondary metabolic complications manifested by abnormal blood levels of glucose, ROS and/or FFA compared to their respective levels in healthy control subjects. Non-limitative examples of such disorders are sepsis and autoimmune diseases.

Certain exemplary disorders in which it may be valuable to assess or monitor the metabolic or glycemic state in accordance with the methods of the invention or by using the kits of the invention are detailed below.

As used herein, the term “reactive oxygen species” (“ROS”) means activated oxygen species such as superoxide (O₂⁻), hydrogen peroxide (H₂O₂), hydroxyl radicals (OH), and singlet oxygen (¹O₂). Excess ROS production in the mitochondria is associated with an unbalanced metabolic state and accompanying pathologies.

Fatty acids are classified according to their length and degree of bond saturation, and may be either free fatty acids (FFA) or fatty acids esterified to other molecules. The term “free fatty acid” is used herein as it is in the art in that FFA are not part of other molecules such as triglycerides or phospholipids (non-esterified fatty acids, NEFA). FFA also include non-esterified fatty acids that are bound to or adsorbed onto albumin. Fatty acids are oxidated by mitochondrial oxidative pathways. Abnormal blood levels of FFA may be found in subject characterized by an unbalanced metabolic state and accompanying pathologies.

In some embodiments, the invention provides for methods and means for determining the glycemic control status of a subject. In other words, the methods and kits of the invention may be used for determining whether the glycemic control status of a subject is balanced (favorable) or unbalanced (unfavorable). An unfavorable or unbalanced glycemic control status is a pathological disease state resulting from (and characterized by) excess blood glucose, which is typically associated with blood glucose levels (e.g. sustained or recurring) that are over 180 mg/dL two hours post prandial and/or HbA1c levels >7%. An unfavorable glycemic control status is often found in unbalanced diabetic patients, and indicates the requirement for medical intervention that would result in blood glucose level normalization (glycemic control balance or homeostasis) and a balanced disease state.

Thus, according to these embodiments of the invention, a UCP2 mRNA level that is significantly higher in platelets of said subject compared to control values, said control values corresponding to its level in platelets of healthy control subjects, indicates an unfavorable glycemic control status in said subject.

In some embodiments, diabetic patients achieving glycemic control (a favorable glycemic control status) may also be characterized by a balanced oxidative status and restoration (or maintenance) of normal reactive oxygen species (ROS) and/or free fatty acids (FFA) levels, as detailed herein. Subjects in which either glycemic control or oxidative status are not balanced are considered to be in an unbalanced metabolic state of the disease.

According to other embodiments, platelet UCP2 mRNA is identified as a prognostic marker for a subject's oxidative status or state. In a subject having an unfavorable oxidative state the oxidative stress is high and the patient may suffer from a state of insulin resistance and abnormal FFA blood values.

“Oxidative stress” generally refers to a pathophysiological state characterized by the generation of ROS in a biological system that exceeds the ability of the system to at least partially neutralize or eliminate them. The imbalance can result from a lack of antioxidant capacity caused by disturbance in production, distribution, or by an overabundance of ROS from an environmental or behavioral stressor. If not regulated properly, the excess ROS can damage the lipids, protein or DNA of a cell, altering its normal function and leading ultimately to the development of certain disease states. The etiology of diseases involving oxidative stress is in part related to the damage caused by the primary and secondary ROS.

As used herein, the term “significantly” as it relates to altered or enhanced blood levels of analytes refers to significant alterations or elevations in accordance with their accepted definitions, as would be recognized by the person skilled in the relevant art. Such levels may differ by e.g. 5%, 10%, 20%, 30%, 40%, 50% or more from the respective control value. With respect to analyte levels for which no such accepted definitions are recognized and unless indicated otherwise, the term refers to values which differ by at least two standards of deviation (SD) from the respective control value.

Disorders

The following exemplary disorders may be evaluated or monitored according to the principles of the invention.

Diabetes

The term “diabetes mellitus” as used herein refers to a disorder of carbohydrate metabolism that is typically characterized by hyperglycemia and glycosuria, which results from inadequate production or utilization of insulin. Diabetes mellitus includes several syndromes or disorders, for example, but not limited to, primary diabetes mellitus (e.g., insulin-dependent (Type I, T1DM) and non-insulin-dependent (Type II, T2DM)); secondary diabetes for example: pancreatic diabetes (e.g., destruction of the pancreas, removal of the pancreas, etc.); extrapancreatic/endocrine diabetes (e.g., hypersomatotropism, hyperadrenalism, hyperthyroidism, glucagonama, etc.); drug-induced diabetes (e.g., steroid diabetes, thiazides, etc.) and rare/exceptional forms of diabetes (e.g., lipoatrophic diabetes, myatonic diabetes, disturbance of insulin receptors, genetic syndromes, etc). Non-balanced diabetic patients typically manifest an unfavorable glycemic control status as defined above and may also manifest an unfavorable metabolic status.

T1DM patients typically require insulin treatment, which may also be helpful for management of many patients with T2DM. A number of analogs, created by modifications of the human insulin molecule that alter subcutaneous absorption rates, are available. Oral antihyperglycemic drugs are the primary treatment for T2DM, although insulin is often added when over two oral drugs fail to provide adequate glycemic control. Such drugs include for example Insulin secretagogues (e.g. Sulfonylureas such as Acetohexamide or short acting such as Nateglinide), Insulin sensitizers (e.g. Biguanides such as Metformin and Thiazolidinediones such as Pioglitazone), α-Glucosidase inhibitors, Dipeptidyl peptidase-4 inhibitors, Glucagon-like peptide-1 agonists and Amylin analogs.

Aberrant Energy Homeostasis and Related Metabolic Disorders

Nutritional disorders include a group of disorders of behavioral and/or environmental etiology, characterized by decreased and/or increased energy intake and consequent imbalance in energy homeostasis. These include inter alia eating disorders ranging from anorexia nervosa on one end and obesity at the other end of the spectrum.

Such disorders are frequently associated with hormonal and metabolic aberrations and pathologies and are often characterized by abnormal (e.g. elevated) blood levels of glucose, ROS and/or FFA.

Significantly decreased caloric intake (as in anorexia nervosa or starvation) is known to lead to malnutrition, metabolic abnormalities as well as changes in immune function, inflammation and hormonal (mainly reproductive) changes. Anorexia nervosa is associated with a significant and occasionally dramatic decrease in food intake resulting in progressive weight loss. This disease state has been defined as a complex syndrome characterized by the diagnostic criteria of a) body weight more than 15% lower than that expected for age and height, b) anxiety and fear of obesity, c) disturbed attitudes towards body weight and shape, and d) hypothalamic amenorrhea for >3 months in post pubertal females. The latter is closely associated with a decrease of body weight to levels below the threshold of about 70% of the mean weight of a matched population. In response to this degree of weight loss, multiple endocrine axes apart from the reproductive axis are also disrupted.

Obesity is a chronic disease and a major health concern in modern society. About 30% adults in U.S. are obese, and about 65% adults are overweight. Obesity is associated not only with a social stigma, but also with decreased life span and is considered a risk factor in developing many health problems, including hypertension; type 2 diabetes mellitus; elevated plasma insulin concentrations; insulin resistance; dyslipidemia; hyperlipidemia; endometrial, breast, prostate and colon cancer; osteoarthritis; respiratory complications, such as obstructive sleep apnea; cholelithiasis; gallstones; arteriosclerosis; heart disease; abnormal heart rhythms; and heart arrhythmias.

Obesity is a condition in which there is an excess of body fat in a subject, which may be due to any cause, whether genetic or environmental. The operational definition of obesity is based on the Body Mass Index (BMI), which is calculated as body weight per height in meters squared (kg/m²). Generally, “Obesity” refers to a condition whereby an otherwise healthy subject has a Body Mass Index (BMI) greater than or equal to 30.0 kg/m², or a condition whereby a subject with at least one co-morbidity has a BMI greater than or equal to 27.0 kg/m². An “obese subject” is an otherwise healthy subject with a Body Mass Index (BMI) greater than or equal to 30.0 kg/m² or a subject with at least one co-morbidity with a BMI greater than or equal to 27.0 kg/m². An obese subject may have a BMI of at least about any of 31.0, 32.0, 33.0, 34.0, 35.0, 36.0, 37.0, 38.0, 39.0, and 40.0. In contrast, an “overweight subject” is a subject with a BMI of 25.0 to 29.9 kg/m².

Syndrome X (or metabolic syndrome) denotes a set of signs and symptoms associated with the accumulation of fat in the abdomen. This form of fat distribution is common in middle-aged men and is often visible as a pot belly or paunch. Syndrome X is characterized by a number of disorders including gout, impaired glucose metabolism (increasing susceptibility to diabetes), raised blood pressure, and elevated blood cholesterol levels. People with Syndrome X have a high risk of heart disease. Syndrome X is defined as a constellation of metabolic abnormalities in serum or plasma insulin/glucose level ratios, lipids, uric acid levels, vascular physiology, and coagulation factor imbalances by the American Association of Clinical Endocrinologists. The term “syndrome X” as used herein thus refers to a condition characterized by positive diagnosis of at least two of the following: Non-insulin-dependent diabetes, blood pressure above a level considered normal, insulin level above a level considered normal, dyslipidemia, and obesity.

Inflammation and Associated Metabolic Aberrations

The invention refers in some embodiments to UCP2 evaluation in inflammatory disorders manifested by metabolic complications resulting in an unfavorable metabolic state. These include systemic inflammatory diseases such as sepsis, autoimmune diseases (e.g. inflammatory bowel disease and rheumatoid arthritis) and other chronic systemic inflammatory diseases.

Sepsis is a potentially deadly medical condition characterized by a whole-body inflammatory state (called a systemic inflammatory response syndrome or SIRS) caused by severe infection. The term “sepsis” as used herein is defined as a SIRS to an infective process in which severe derangement of the host immune system fails to prevent extensive “spill over” of inflammatory mediators from a local infection focus into the systemic circulation. Common symptoms of sepsis include those related to a specific infection, but usually accompanied by high fevers, hot, flushed skin, elevated heart rate, hyperventilation, altered mental status, swelling, and low blood pressure. In the very young and elderly, or in people with weakened immune systems, the pattern of symptoms may be atypical, with hypothermia and without an easily localizable infection. Sepsis is usually treated with intravenous fluids and antibiotics, however some might benefit from tight control of blood sugar levels with insulin (targeting stress hyperglycemia).

Autoimmune diseases arise from an inappropriate immune response of the body against substances and tissues normally present in the body (autoimmunity). While autoimmunity may be restricted to certain organs or involve a particular tissue in different places, embodiments of the invention relate to prognosing and monitoring subjects afflicted with autoimmune diseases which include systemic autoimmune reactions, e.g. rheumatoid arthritis, T1DM and inflammatory bowel disease (IBD). The treatment of autoimmune diseases is typically with immunosuppression—medication that decreases the immune response.

Inflammatory bowel disease (IBD), a form of chronic gastrointestinal inflammation, includes a group of chronic inflammatory disorders of generally unknown etiology, including ulcerative colitis (UC) and Crohn's disease (CD). These diseases appear to result from the unrestrained activation of an inflammatory response in the intestines. This inflammatory cascade is thought to be perpetuated through the actions of pro-inflammatory cytokines and selective activation of lymphocyte subsets. In patients with IBD, ulcers and inflammation of the inner lining of the intestines lead to symptoms of abdominal pain, diarrhea, and rectal bleeding. Ulcerative colitis occurs in the large intestine, while in Crohn's the disease can involve the entire GI tract, both small and large intestines. UC is a condition that primarily affects the superficial layer of the colon mucosa and histological analyses reveal ulceration of the mucosa, blunting and loss of crypts, and an inflammatory infiltrate.

Treatment of IBD commonly utilizes a variety of orally administered systemic anti-inflammatory agents designed to reduce the inflammatory response. First line therapy commonly employs 5-aminosalicylate (Mesalamine), or 5-aminosalicylate precursors, such as sulfasalazine, olsalazine, or balsalazide, immunosuppressive agents, such as cyclosporine, azathioprine, and 6-mercaptopurine, corticosteroids such as beclometasone or budesonide and biologics such as infliximab, anti-leukocyte adhesions molecules, and daclizumab. Due to the postulated role of bacterial infection in IBD, eradication of the gut bacterial flora is also attempted by means that include use of antibiotics and antimicrobial agents. About 20-25% of the patients with UC fail to respond to medical therapy and therefore are referred to surgery for total proctocolectomy. In general, patients with CD are less responsive to medical therapy and usually do not respond to surgical treatment.

Rheumatoid arthritis (RA, rheumatic disease) is an autoimmune chronic disease marked by stiffness and inflammation of the joints, weakness, loss of mobility, and deformity. The process involves an inflammatory response of the capsule around the joints (synovium) secondary to swelling (turgescence) of synovial cells, excess synovial fluid, and the development of fibrous tissue (pannus) in the synovium. The pathology of the disease process often leads to the destruction of articular cartilage and ankylosis (fusion) of the joints. RA can also produce diffuse inflammation in the lungs, the membrane around the heart (pericardium), the membranes of the lung (pleura), and white of the eye (sclera), and also nodular lesions, most common in subcutaneous tissue. Although the cause of RA is unknown, autoimmunity plays a big part, and RA is a systemic autoimmune disease. It is a clinical diagnosis made on the basis of symptoms, physical exam, radiographs (X-rays) and labs.

Treatments are pharmacological and non-pharmacological. Non-pharmacological treatment includes physical therapy, orthoses, occupational therapy and nutritional therapy but these don't stop the progression of joint destruction. Analgesia (painkillers) and anti-inflammatory drugs, including steroids, suppress symptoms, but don't stop the progression of joint destruction either. Disease-modifying antirheumatic drugs (DMARDs) slow or halt the progress of the disease.

Chronic obstructive pulmonary disease (COPD) is partially reversible airflow limitation caused by an inflammatory response to inhaled toxins, often cigarette smoke. α1-Antitrypsin deficiency and various occupational exposures are less common causes in nonsmokers. Symptoms are productive cough and dyspnea that develop over years; common signs include decreased breath sounds, prolonged expiratory phase of respiration, and wheezing. Severe cases may be complicated by weight loss, pneumothorax, frequent acute decompensation episodes, right heart failure, and acute or chronic respiratory failure. Diagnosis is based on history, physical examination, chest x-ray, and pulmonary function tests. Treatment is with bronchodilators, corticosteroids, and, when necessary, O₂ and antibiotics. About 50% of patients die within 10 years of diagnosis.

Endocrine Imbalance and Consequent Metabolic Aberrations

In other embodiments, UCP2 evaluation in accordance with the invention may be performed in subjects afflicted with endocrine disorders associated with an unfavorable metabolic state. For example, aberrant expression or function of thyroid hormones (e.g. thyroxine, triiodothyronine) or thyroid-stimulating hormones (TSH), having a metabolism-stimulating activity, may result in the development of disorders associated with an unbalanced metabolic state. In some patients receiving thyroid hormones or TSH, metabolic state may still be unbalanced despite seemingly normal hormone levels as appearing in lab tests, or alternatively, appear to have aberrant thyroid/TSH hormone levels while the general metabolic state has resumed to a balanced state. Thus, additional prognostic means for monitoring these patients for treatment compatibility are needed.

The thyroid is an endocrine gland situated in the neck, which takes up iodine from the bloodstream and synthesizes two hormones, tri-iodothyronine (T3) and thyroxine (T4, tetraiodothyronine). The active hormone is T3, which controls the basal metabolic rate; thyroxine is converted to T3 in tissues by the action of a selenium-dependent enzyme. Thyroid hormone acts as a catalyst for oxidative processes of the body cell and thus regulates the rates of body metabolism and stimulates body growth and maturation.

Disorders associated with hypothyroidism may be e.g. of autoimmune, genetic or surgical etiologies.

Hashimoto's thyroiditis or chronic lymphocytic thyroiditis is an autoimmune disease in which the thyroid gland is attacked by a variety of cell- and antibody-mediated immune processes. Hashimoto's thyroiditis typically results in hypothyroidism with bouts of hyperthyroidism. Symptoms of Hashimoto's thyroiditis include Myxedematous psychosis, weight gain, depression, mania, sensitivity to heat and cold, paresthesia, fatigue, panic attacks, bradycardia, tachycardia, high cholesterol, reactive hypoglycemia, constipation, migraines, muscle weakness, cramps, memory loss, infertility and hair loss.

The thyroid gland may become firm, large, and lobulated in Hashimoto's thyroiditis, but changes in the thyroid can also be nonpalpable. Enlargement of the thyroid is due to lymphocytic infiltration and fibrosis rather than tissue hypertrophy. Physiologically, antibodies against thyroid peroxidase (TPO) and/or thyroglobulin cause gradual destruction of follicles in the thyroid gland. Accordingly, the disease can be detected clinically by looking for these antibodies in the blood. It is also characterized by invasion of the thyroid tissue by leukocytes, mainly T-lymphocytes. It is associated with non-Hodgkin lymphoma.

Hypothyroidism may also result from thyroidectomy, i.e. surgical removal of the thyroid gland, e.g. in case of thyroid cancer or some other condition of the thyroid gland (such as hyperthyroidism) or goiter. After the removal of a thyroid, patients usually take a prescribed oral synthetic thyroid hormone—levothyroxine (Synthroid)—as a countermeasure for hypothyroidism. The dosing schedule of the thyroid hormone replacement therapy often needs to be adjusted throughout the patient's life to ascertain proper metabolic balance.

Hyperthyroidism typically results from the development of autoimmune processes, although environmental and other causes are also known. Graves' disease is an autoimmune disease affecting the thyroid, frequently causing it to enlarge to twice its size or more (goiter), become overactive, with related hyperthyroid symptoms such as increased heartbeat, muscle weakness, disturbed sleep, and irritability. It can also affect the eyes, causing bulging eyes (exophthalmos). It affects other systems of the body, including the skin, heart, circulation and nervous system.

According to some embodiments, the invention further relates to methods and means for prognosing or monitoring metabolic or inflammatory disorders that do not involve platelet dysfunction or thrombocytopenia.

Sample and Platelet Isolation and Purification

Methods for obtaining and isolating platelets are known in the art. Whole blood samples may be collected from the subject of interest into a suitable anticoagulant (e.g. ethylenediaminetetraacetate (EDTA) or acid citrate dextrose) as known in the art. Platelets may be separated or isolated from whole blood using methods commonly used in the art, e.g. by one or more techniques including filtration, density fractionation methods such as centrifugation of whole blood, centrifugation of blood in multiple stages, and continuous-flow centrifugation, or by using antibody-based methods to deplete unwanted blood components.

In one embodiment, a unit of whole blood is centrifuged using settings that precipitate only the cellular components of the blood (e.g., red blood cells and white blood cells). At these settings, the platelets remain suspended in the plasma. The platelet-rich plasma (PRP) is removed from the precipitated blood cells, and then centrifuged at a faster setting to harvest the platelets from the plasma.

In another embodiment, the whole blood is centrifuged using settings that cause the platelets to become suspended in the “buffy coat” layer, which includes the platelets and the white blood cells. The “buffy coat” is isolated in a sterile bag, suspended in a small amount of red blood cells and plasma, then centrifuged again to separate the platelets and plasma from the red and white blood cells.

In another embodiment, apheresis platelets are collected using a mechanical device that draws blood from the donor and centrifuges the collected blood to separate out the platelets and other components to be collected. The remaining blood is returned to the donor.

Various devices that can assist in platelet separation are also available. Embodiments of such devices used for isolating platelet-rich plasma include the GPS™ II Platelet Concentrate Separation Kit and the Plasmax™ Plus Plasma Concentrator accessory (Biomet Biologics, Inc., Warsaw, Ind.). Such devices and methods are described in U.S. Patent Application Publication 2004/0251217 (Leach et al.), and U.S. Patent Application Publication 2005/0109716 (Leach et al.), which are hereby incorporated by reference. Another example of a device that may be used in isolating platelet-rich plasma by density fractionation includes a centrifugal drum separator and an erythrocyte capture trap. In one embodiment, the walls of the centrifugal drum separator are coated with a depth filter having pores and passageways that are sized to receive and entrap erythrocytes. Blood is placed in the centrifugal drum, and the drum is spun along its axis at sufficient speed so as to force erythrocytes from the blood into the depth filter. After spinning, the erythrocytes remain in the filter and the remaining platelet-rich plasma is extracted. The platelet-rich plasma may be concentrated by desiccation. Embodiments of such devices include the Vortech™ Concentration System (Biomet Biologics, Inc., Warsaw, Ind.), and are disclosed in U.S. Patent Application Publication 2006/0175244 (Dorian et al.) and U.S. Patent Application Publication 2006/0175242 (Dorian et al.), which are hereby incorporated by reference. Such devices may be used to prepare platelet-rich plasma in lieu of or in addition to using the tube having a buoy. Other devices for isolating platelet-rich plasma use high speed centrifugation to pellet the platelets and red blood cells. The pelleted platelets are then re-suspended using some of the plasma supernatant or another suitable solution. It will be understood, however, that other suitable methods for forming the platelet-rich plasma may also be used.

According to certain embodiments of the invention, UCP2 mRNA levels are determined in at least partially purified platelets, which may be isolated from a blood sample obtained from the subject. At least partly purified or isolated platelets denote platelet-rich preparations as accepted in the art and/or preparations obtained as described above. According to certain advantageous embodiments, contamination of the platelet preparation by other cell types such as red blood cells and leukocytes is minimized, in order to assure that mRNA levels adequately represent their levels in platelets. In some embodiments, this is especially important for leukocytes, which have been reported to contain as much as 10,000 times more RNA than platelets. Thus, according to certain embodiments, the methods and kits of the invention are effected using at least partly purified platelets that are at least 90%, at least 95%, at least 98% or about 100% pure. Platelet preparations which are about 100% pure (free of other blood cells) are considered as isolated from other blood components.

Platelets are anuclear and as a consequence, all non-mitochondrial platelet mRNA is derived from their precursor cell, the megakaryocyte. Accordingly, the amount of mRNA a platelet contains depends on the amount of mRNA inherited when it budded off from the megakaryocyte as well as the rate of platelet RNA degradation. According to certain embodiments, a fast platelet purification method can help to minimize further degradation of mRNAs during the purification process.

Thus, according to some embodiments, the methods of the invention preferably assay mRNA levels in freshly isolated platelets, namely platelets not cultured, incubated or otherwise maintained ex vivo for over 2, 3, 5, 7 or 9 hours. Typically, RNA levels are assayed in embodiments of the invention within about 2-5 hours of platelet isolation. Methods for obtaining suitable samples and isolating (or enriching a sample with) platelets and mRNA are well known in the art (see e.g. Examples). By way of a non limiting example, embodiments of the invention may utilize rapid platelet isolation methods as described by Amisten et al. (2012). Briefly, Amisten et al. describe a method for isolating human circulating platelets from small volumes of whole blood based on efficient inhibition of platelet activation and leukocyte removal by filtration followed by antibody-mediated magnetic bead-depletion of residual contaminating leukocytes and red blood cells (using e.g. Dynabead® system, Invitrogen).

According to certain other embodiments, platelet activation is inhibited to minimize alteration in gene expression which may be induced or accelerated ex vivo. This may be effected using known techniques e.g. using PGE1 and/or apyrase as exemplified herein.

The experimental examples below demonstrate several non-limitative examples for platelet isolation involving cell sedimentation by e.g. centrifugation of anti-coagulated blood, wherein the preparation may be treated by PGE1 and apyrase to reduce platelets' activation induced by in-vitro manipulations.

Assays and Kits

Determining, measuring or quantifying the level of UCP2 mRNA and UCP2 protein may be performed by a variety of methods well known in the art. For example, the methods and assays of the invention may include, without limitation, the use of: RT-PCR, real-time RT-PCR, Oligonucleotide microarray or Northern blot. Each possibility represents a separate embodiment of the invention.

Isolation of RNA from platelets and optional mRNA purification may be performed by conventional methods known in the art (see e.g. Sambrook, J. and Russell, D. W. (2001), Molecular Cloning: A Laboratory Manual). Commercial kits for RNA extraction are also available, e.g. EZ-RNA (Biological Industry, Kibbutz Beit Haemek) and TRIZOL® (Gibco-BRL™, USA).

The expression level of the RNA in platelets can be determined using methods known in the art; a brief description of non-limiting examples of such methods is provided hereinbelow.

RT-PCR and Real Time RT-PCR Analyses

Reverse transcriptase-polymerase chain reaction (RT-PCR) uses PCR amplification of relatively rare RNA molecules. First, RNA molecules are purified from cells and converted into complementary DNA (cDNA) using a reverse transcriptase enzyme (such as an MMLV-RT) and primers such as oligo-dT, random hexamers, or gene-specific primers. Then by applying gene-specific primers and Taq DNA polymerase, a PCR amplification reaction is carried out in a PCR machine. Those of ordinary skill in the art are capable of selecting the length and sequence of the gene-specific primers and the PCR conditions (i.e., annealing temperatures, number of cycles, and the like) that are suitable for detecting specific RNA molecules. It will be appreciated that a semi-quantitative RT-PCR reaction can be employed, by adjusting the number of PCR cycles and comparing the amplification product to known controls.

Real time RT-PCR follows the general principle of RT-PCR, with the difference being that the amplification product is measured in “real time” during the PCR reaction rather than at the end of the process. This enables the researcher to observe the amplification before any reagent becomes rate limiting for amplification.

Two common methods for detection of products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary DNA target.

Several fluorescence methodologies are available to measure amplification product in real-time PCR. Taqman (Applied BioSystems, Foster City, Calif.) uses fluorescence resonance energy transfer (FRET) to inhibit signal from a probe until the probe is degraded by the sequence specific binding and Taq 3′ exonuclease activity. Molecular Beacons (Stratagene, La Jolla, Calif.) also use FRET technology, whereby the fluorescence is measured when a hairpin structure is relaxed by the specific probe binding to the amplified DNA. The third commonly used chemistry is Sybr Green, a DNA-binding dye (Molecular Probes, Eugene, Oreg.). The more amplified product that is produced, the higher the signal. Other detection chemistries can also been used, such as ethidium bromide or other DNA-binding dyes and many modifications of the fluorescent dye/quencher dye Taqman chemistry, for example scorpions.

Oligonucleotide Microarray

In this method, oligonucleotide probes capable of specifically hybridizing with the polynucleotides of interest, e.g. the UCP2 mRNA, are attached to a solid surface (e.g., a glass wafer). Each oligonucleotide probe is of approximately 20-25 nucleic acids in length. To detect the expression pattern of the polynucleotides of the present invention in a specific sample (e.g., platelets), RNA is extracted from the cell sample using methods known in the art (using, e.g., a TRIZOL® solution, Gibco-BRL™, USA). Hybridization can take place using either labeled oligonucleotide probes (e.g., 5′-biotinylated probes) or labeled fragments of complementary DNA (cDNA) or RNA (cRNA). Briefly, double-stranded cDNA is prepared from the RNA using reverse transcriptase (RT) (e.g., Superscript™ II RT), DNA ligase, and DNA polymerase I, all according to the manufacturer's instructions (Invitrogen Life Technologies, Frederick, Md., USA). To prepare labeled cRNA, the double-stranded cDNA is subjected to an in vitro transcription reaction in the presence of biotinylated nucleotides using, e.g., the BioArray™ HighYield™ RNA Transcript Labeling Kit (Enzo Diagnostics, Inc., Farmingdale, N.Y., USA). For efficient hybridization the labeled cRNA can be fragmented e.g. by incubating the RNA in 40 mM Tris Acetate (pH 8.1), 100 mM potassium acetate, and 30 mM magnesium acetate, for 35 minutes at 94° C. Following hybridization, the microarray is washed and the hybridization signal is scanned using a confocal laser fluorescence scanner, which measures fluorescence intensity emitted by the labeled cRNA bound to the probe arrays.

For example, in the Affymetrix® GeneChip® Microarray (Affymetrix, Inc., Santa Clara, Calif., USA), each gene on the array is represented by a series of different oligonucleotide probes, of which each probe pair consists of a perfect-match oligonucleotide and a mismatch oligonucleotide. While the perfect-match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent DNA Microarray Scanner™ (Agilent Technologies, USA) and the Microarray Suite™ (MAS) (Affymetrix, Inc.) software subtracts the non-specific signal of the mismatch probe from the signal resulting from the perfect-match probe.

Northern Blot Analysis

This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radioisotopes or enzyme-linked nucleotides. Detection may be performed by autoradiography, colorimetric reaction, or chemiluminescence. This method allows for both quantitation of an amount of a particular RNA molecule and determination of its identity by a relative position on the membrane which is indicative of a migration distance in the gel during electrophoresis.

Primer and probe sequences to be used in the above and other assays may be readily prepared by the skilled artisan based on UCP2 sequences known in the art. In a particular embodiment the UCP2 is human UCP2. Non-limitative examples of such sequences are exemplified below (see e.g. SEQ ID NOs: 1-2 and 3-6 below). Primers can be selected manually by the skilled artisan, or with the assistance of a computer implemented algorithm that optimizes primer selection based on desired parameters, such as annealing temperature, length, GC content, etc. Numerous computer implemented algorithms or programs for use via the internet or on a personal computer are available for these purposes, for example CODEHOP, DoPrimer, Primer3, Primer Selection, Web Primer, PCR Designer and others.

In another aspect, there is provided a kit (or assay) comprising means for determining the level of UCP2 mRNA, and at least one of: i) means for isolating platelets from a sample and ii) instructions for determining the level of UCP2 mRNA in isolated platelets. The kit may optionally further comprise a control sample or data useful for comparing the level of UCP2 mRNA to its level in healthy control subject(s).

In certain embodiments, means for determining the level of UCP2 mRNA may include for example the reagents or systems disclosed above as suitable for RNA level evaluation. For example, these may include PCR primers or probes that may be used in real-time RT-PCR, microarray or Northern blot techniques, optionally in combination with one or more enzymes, buffers or other reagents used in these methods.

In other embodiments, means for isolating platelets from a sample may include e.g. reagents and kits as described above for platelet isolation and purification from a blood sample. It is to be understood, that since some of these methods employ the use of reagents that may be used for other purposes, the means for isolating platelets from a sample referred to herein include reagents or reagent combinations recognized to be used for platelet isolation, preferably wherein these reagents or combinations are not regularly used for other purposes, and/or wherein the kit further includes instructions for determining the level of UCP2 mRNA in isolated platelets. For example, without limitation, the kit may contain antibody-conjugated magnetic beads for depleting erythrocytes and lymphocytes, optionally with reagents such as PGE1 and apyrase for inhibiting platelet activation, with instructions for using these reagents for platelet isolation.

The kit may optionally further comprise a control sample or data useful for comparing the level of UCP2 mRNA to its level in healthy control subject(s). The kit may optionally contain a positive control sample of UCP2 cDNA and/or a negative control cDNA sample of an irrelevant gene transcript (e.g. actin). The kit may optionally comprise a set of stored data with average values or ranges of values representative of UCP2 mRNA in platelets of healthy control subjects (or balanced diabetic subjects exhibiting a favorable glycemic control/metabolic status) and/or of subjects with an unfavorable glycemic control/metabolic status subjects e.g. of unbalanced diabetic subjects, measured using the reagents and instructions of the kit.

The kits of the invention may be used in various embodiments for determining the glycemic control status of a subject, for determining the metabolic status of a subject or for evaluating or selecting a treatment to a disorder in a subject in need thereof, as detailed herein.

Methods

According to a first aspect of the invention, there is provided a method for determining the glycemic control status of a subject, comprising determining the level of Uncoupling Protein-2 (UCP2) mRNA specifically in platelets of said subject. By “determining the level of UCP2 mRNA specifically in platelets of said subject” it is meant that the level of UCP2 mRNA is determined in at least partially purified platelets of said subject as detailed herein compared to control values. In other words, the UCP2 mRNA level in platelets of said subject is compared to their levels in platelets taken from suitable control subjects, for example healthy subjects or subjects having a favorable glycemic control status, as detailed herein. It is to be understood, that other than their source, the test platelets and the control platelets to which they are compared should be otherwise equivalent in terms of their degree of purity, ex-vivo activation level etc’, and are preferably obtained, purified and maintained by the same processes. Thus when isolated or partially purified platelets are tested the appropriate control reflects the level in control platelets isolated or at least partially purified in the same manner.

In another embodiment there is provided an in vitro method for determining the glycemic control status of a subject, comprising determining the level of Uncoupling Protein-2 (UCP2) mRNA specifically in platelets of said subject.

In another embodiment, the platelets are isolated from a blood sample obtained from the subject.

In another embodiment, a platelet UCP2 mRNA level that is significantly higher than its level in healthy control subjects indicates an unfavorable glycemic control status in said subject.

It is to be understood, that the levels of mRNA in the platelets, including UCP2 mRNA, reflects, at most, the status 10-14 days prior to the time the platelets are isolated from a subject (corresponding to the time the blood sample is obtained). Thus in another embodiment, said method is used for determining the glycemic control status of said subject within the preceding 5-14 days, wherein each possibility represents a separate embodiment of the invention. Thus, the platelet UCP2 mRNA levels may be used to determine the glycemic control status within the preceding 5-12, 5-10, 5-7, 7-9, 7-11 or 7-14 days. In another embodiment said method is used for determining the glycemic control status of said subject within the preceding 5-7 days of platelet isolation.

In another embodiment the subject is afflicted with diabetes mellitus. In a particular embodiment said subject is afflicted with type II diabetes mellitus.

In another embodiment said method further comprises assessing the metabolic status of the subject, wherein a platelet UCP2 mRNA level that is significantly higher than its level in healthy control subjects indicates an unfavorable metabolic status in said subject.

In another embodiment, the methods may be performed by using a method comprising:
a) obtaining platelets from the subject (e.g. from a blood sample of said subject);
b) determining the level of UCP2 mRNA specifically in the platelets;
c) comparing the level of UCP2 mRNA to a control UCP2 mRNA level (e.g. the level in platelets of healthy control subject(s), in a panel of control samples from a set of healthy or balanced individuals, in a stored set of data from control healthy or balanced individuals, or in a sample previously obtained from the same subject during a balanced state of the disease);
   wherein a level of UCP2 mRNA that is significantly (statistically significant or significant to one of skill in the art) elevated compared to the control level indicates that the subject has an unfavorable or unbalanced glycemic control status.

In another aspect there is provided a method for determining the metabolic status of a subject, comprising determining the level of Uncoupling Protein-2 (UCP2) mRNA specifically in platelets of said subject. In another embodiment there is provided an in vitro method for determining the metabolic status of a subject, comprising determining the level of Uncoupling Protein-2 (UCP2) mRNA specifically in platelets of said subject.

In another embodiment the platelets are isolated from a blood sample obtained from the subject.

In another embodiment a platelet UCP2 mRNA level that is significantly higher than its level in healthy control subjects indicates an unfavorable metabolic status in said subject.

In another embodiment the subject is afflicted with a chronic disorder associated with abnormal (e.g. elevated) blood levels of glucose, reactive oxygen species (ROS) or free fatty acids (FFA). In another embodiment the subject is afflicted with a chronic disorder associated with elevated blood levels of glucose, ROS and FFA. According to certain particular embodiments, the disorder may include, without limitation, diabetes mellitus, a nutritional disorder, a chronic inflammatory disease (associated with metabolic aberrations) and an endocrine disease. Each possibility represents a separate embodiment of the invention. For example, diabetes mellitus may include type I diabetes and type II diabetes; the nutritional disorder may include anorexia nervosa, obesity and syndrome X; the chronic inflammatory disease may include sepsis, autoimmune diseases (e.g. inflammatory bowel disease (IBD), type 1 diabetes, Hashimoto's thyroiditis, Graves' disease and rheumatoid arthritis (RA)) and chronic obstructive pulmonary disease (COPD)); and the endocrine disease may include disorders associated with hypothyroidism (e.g. Hashimoto's thyroiditis), and disorders associated with hyperthyroidism (e.g. Graves' disease). Each possibility represents a separate embodiment of the invention. According to other particular embodiments, the disorder is selected from the group consisting of type I diabetes, type II diabetes, anorexia nervosa, obesity, syndrome X, sepsis, IBD, RA, COPD, Hashimoto's thyroiditis and Graves' disease, wherein each possibility represents a separate embodiment of the invention. In another embodiment the disorder is selected from the group consisting of type I diabetes, type II diabetes, anorexia nervosa, obesity, syndrome X, sepsis, IBD, RA, Hashimoto's thyroiditis and Graves' disease. According to other particular embodiments, the disorder is selected from the group consisting of type I diabetes, type II diabetes, anorexia nervosa, sepsis, hypothyroidism, IBD, COPD and rheumatic disease (RA). In another embodiment, the disorder is associated with aberrations in glucose metabolism. Such disorders may involve the development of insulin resistance, and are typically manifested by elevated blood levels of glucose. Examples for such disorders include, without limitation, diabetes mellitus (e.g. T1DM and T2DM), hypothyroidism associated disorders, Syndrome X and obesity. According to yet another particular embodiment, said disorder is diabetes mellitus. According to yet another particular embodiment, said disorder is type II diabetes mellitus.

In another embodiment said method is used for determining the metabolic status of said subject within the preceding 5-14 days of platelet isolation. In another embodiment said method is used for determining the metabolic status of said subject within the preceding 5-7 days of platelet isolation. Thus, the platelet UCP2 mRNA levels may be used to determine the metabolic status within the preceding 5-12, 5-10, 5-7, 7-9, 7-11 or 7-14 days.

In another embodiment, and the method comprises determining the glycemic control status of a subject, wherein a platelet UCP2 mRNA level that is significantly higher than its level in healthy control subjects indicates an unfavorable glycemic control status in said subject.

In another embodiment the subject is afflicted with type II diabetes mellitus, and the method further comprises determining the glycemic control status of the subject, wherein a platelet UCP2 mRNA level that is significantly higher than its level in healthy control subjects indicates an unfavorable glycemic control status in said subject.

In another embodiment the subject is receiving a treatment for the disorder, wherein a platelet level of UCP2 mRNA that is significantly elevated compared to its level in healthy control subjects indicates that the treatment is unsuitable for said subject.

In another embodiment, the methods may be performed by using a method comprising:
a) obtaining platelets from the subject (e.g. from a blood sample of said subject);
b) determining the level of UCP2 mRNA specifically in the platelets;
c) comparing the level of UCP2 mRNA to a control UCP2 mRNA level (e.g. the level in platelets of healthy control subject(s), in a panel of control samples from a set of healthy or balanced individuals, in a stored set of data from control healthy or balanced individuals, or in a sample previously obtained from the same subject during a balanced state of the disease);
   wherein a level of UCP2 mRNA that is significantly (statistically significant or significant to one of skill in the art) elevated compared to the control level indicates that the subject has an unfavorable or unbalanced metabolic status.

In another aspect, there is provided a method for evaluating or selecting a treatment to a disorder in a subject in need thereof, wherein the subject is receiving the treatment for the disorder, and wherein a platelet level of UCP2 mRNA that is significantly elevated compared to its level in healthy control subjects indicates that said treatment is unsuitable for said subject. In another embodiment there is provided an in vitro method for evaluating or selecting a treatment to a disorder in a subject in need thereof, wherein the subject is receiving the treatment for the disorder, and wherein a platelet level of UCP2 mRNA that is significantly elevated compared to its level in healthy control subjects indicates that said treatment is unsuitable for said subject.

In one embodiment, the disorder is a chronic disorder associated with abnormal (e.g. elevated) blood levels of glucose, reactive oxygen species (ROS) or free fatty acids (FFA). In another embodiment the subject is afflicted with a chronic disorder associated with elevated blood levels of glucose, ROS and FFA. According to certain particular embodiments, the disorder may include, without limitation, diabetes mellitus, a nutritional disorder, a chronic inflammatory disease (associated with metabolic aberrations) and an endocrine disease. Each possibility represents a separate embodiment of the invention. For example, diabetes mellitus may include type I diabetes and type II diabetes; the nutritional disorder may include anorexia nervosa, obesity and syndrome X; the chronic inflammatory disease may include sepsis, autoimmune diseases (e.g. inflammatory bowel disease (IBD), type 1 diabetes, Hashimoto's thyroiditis, Graves' disease and rheumatoid arthritis (RA)) and chronic obstructive pulmonary disease (COPD)); and the endocrine disease may include disorders associated with hypothyroidism (e.g. Hashimoto's thyroiditis), and disorders associated with hyperthyroidism (e.g. Graves' disease). Each possibility represents a separate embodiment of the invention. According to other particular embodiments, the disorder is selected from the group consisting of type I diabetes, type II diabetes, anorexia nervosa, obesity, syndrome X, sepsis, IBD, RA, COPD, Hashimoto's thyroiditis and Graves' disease, wherein each possibility represents a separate embodiment of the invention. In another embodiment the disorder is selected from the group consisting of type I diabetes, type II diabetes, anorexia nervosa, obesity, syndrome X, sepsis, IBD, RA, Hashimoto's thyroiditis and Graves' disease. According to other particular embodiments, the disorder is selected from the group consisting of type I diabetes, type II diabetes, anorexia nervosa, sepsis, hypothyroidism, IBD, COPD and rheumatic disease (RA). In another embodiment, the disorder is associated with aberrations in glucose metabolism. Examples for such disorders include, without limitation, diabetes mellitus (e.g. T1DM and T2DM), hypothyroidism associated disorders, Syndrome X and obesity. According to yet another particular embodiment, said disorder is diabetes mellitus. According to yet another particular embodiment, said disorder is type II diabetes mellitus.

In another embodiment the method is used for evaluating the treatment within 2-16 weeks from the onset of said treatment. In another embodiment the method is used for evaluating the treatment at least 2 weeks from the onset of said treatment. In another embodiment said method is used for evaluating the treatment within the preceding 5-14 days of platelet isolation. In another embodiment said method is used for evaluating the treatment within the preceding 5-7 days of platelet isolation.

### Additional Embodiments

The methods and assays of the invention comprise determining (or means for determining) the level of mRNA of a gene product selected from uncoupling protein 2 (UCP2) and uncoupling protein 3 (UCP3) in platelets of said subject. Preferably, the methods and assays comprise determining (or means for determining) the level of UCP2 mRNA in isolated platelets of said subject.

Thus, according to a first aspect of the present invention, there is provided a method for determining the glycemic control status of a subject, comprising determining the level of UCP2 mRNA in at least partially purified platelets of said subject compared to control values. According to some embodiments the platelets are at least partially purified or isolated from other blood components. In another embodiment the platelets are isolated from a blood sample obtained from the subject.

According to embodiments of the invention, a UCP2 mRNA level that is significantly higher than its level in isolated platelets of healthy control subjects indicates an unfavorable glycemic control status in said subject. In some embodiments, diabetic patients achieving glycemic control (a favorable glycemic control status) may also characterized by a balanced oxidative status and restoration of normal ROS and/or FFA levels, as detailed herein.

As the life span of platelets is ten days and they do not have a nucleus, the mRNA in the platelets, including UCP2 mRNA, reflects, at most, the status 10-14 days prior to the date of measurement; typically the mRNA in platelets will reflect the metabolic status at 5-7 days prior to the measurement. Thus, according to some embodiments, the methods and assays of the invention are used for determining the glycemic control status of said subject within the preceding 5-14 days prior to platelet isolation. In other embodiments, said methods and assays are used for determining the glycemic control status of said subject within the preceding 5-7 days prior to platelet isolation. Thus in another embodiment, said method is used for determining the glycemic control or metabolic status of said subject within the preceding 5-14 days, wherein each possibility represents a separate embodiment of the invention. Thus, the platelet UCP2 mRNA levels may be used to determine the glycemic control status within the preceding 5-12, 5-10, 5-7, 7-9, 7-11 or 7-14 days.

In another embodiment, the subject is afflicted with diabetes mellitus. In a particular embodiment, said subject is afflicted with type II diabetes mellitus.

According to other embodiments, the methods and assays of the invention are useful for determining the oxidative state of the subject, wherein a UCP2 mRNA level that is significantly higher than its level in healthy control subjects indicates an unfavorable oxidative state in said subject. In other embodiments, the methods and assays of the invention can also apply to other situations where the concentration of free fatty acids increase in the blood, the oxidative stress is high and the patients suffer from a state of insulin resistance. For example, without limitation, such clinical situations include: diabetes (types I and II), starvation (anorexia nervosa), sepsis, hypothyroidism, inflammatory bowel disease (IBD), chronic obstructive pulmonary disease (COPD), Rheumatic disease and other chronic inflammatory or metabolic diseases. Each possibility represents a separate embodiment of the invention.

Thus, in another aspect, the invention provides a method for determining the metabolic status of a subject, comprising determining the level of mRNA selected from UCP2 and UCP3 mRNA in at least partially purified platelets of said subject compared to control values. According to some embodiments the platelets are isolated from other blood components. In another embodiment the platelets are isolated from a blood sample obtained from the subject.

In a specific embodiment, the method comprises determining the level of UCP2 mRNA in isolated platelets of said subject. In one embodiment, a UCP2 (or UCP3) mRNA level that is significantly higher than its level in isolated platelets of healthy control subjects indicates an unfavorable metabolic status in said subject. An unfavorable metabolic status as used herein refers to blood levels of glucose, ROS and/or FFA that are significantly enhanced compared to healthy control subjects, as detailed herein.

In some embodiments, the subject is afflicted with a chronic disorder associated with elevated blood levels of glucose, ROS or FFA. In other embodiments, the subject is afflicted with a chronic disorder associated with elevated blood levels of glucose, ROS and FFA. In other embodiments, the disorder is selected from the group consisting of type I diabetes, type II diabetes, anorexia nervosa, sepsis, hypothyroidism, IBD, COPD and rheumatic disease. Each possibility represents a separate embodiment of the invention.

For example, the methods and assays of the invention may be performed by using a method comprising:
a) obtaining platelets from the subject (e.g. from a blood sample of said subject);
b) determining the level of UCP2 mRNA in the platelets;
c) comparing the level of UCP2 mRNA to a control UCP2 mRNA level (e.g. the level in platelets of healthy control subject(s), in a panel of control samples from a set of individuals, in a stored set of data from control individuals, or in a sample previously obtained from the same subject during a balanced state of the disease);
   wherein a level of UCP2 mRNA that is significantly (statistically significant or significant to one of skill in the art) elevated compared to the control level indicates that the subject has an unfavorable or unbalanced glycemic control or metabolic state.

According to some embodiments the platelets are at least partially purified or isolated. According to some embodiments the mRNA is isolated from the platelets. The methods of the invention may further comprise the step of evaluating or selecting a therapy for said subject. For example, a level of UCP2 mRNA that is significantly elevated compared to the control level (e.g. after 5-7 days of the onset of a treatment) indicates that the treatment is unsuitable for said subject.

Platelets are anuclear and as a consequence, all non-mitochondrial platelet mRNA is derived from their precursor cell, the megakaryocyte. Accordingly, the amount of mRNA a platelet contains depends on the amount of mRNA inherited when it budded off from the megakaryocyte as well as the rate of platelet RNA degradation. According to preferred embodiments, a fast platelet purification method can help to minimize further degradation of mRNAs during the purification process.

According to some embodiments, the methods of the invention preferably assay mRNA levels in freshly isolated platelets, namely platelets not cultured, incubated or otherwise maintained ex vivo for over 2, 3, 5, 7 or 9 hours. Typically, RNA levels are assayed in embodiments of the invention within about 2-5 hours of platelet isolation. Methods for obtaining suitable samples and isolating (or enriching a sample with) platelets and mRNA are well known in the art (see e.g. Examples). By way of a non limiting example, embodiments of the invention may utilize rapid platelet isolation methods as described by Amisten et al. (2012).

Determining, measuring or quantifying the level of UCP2 mRNA and UCP2 protein may be performed by a variety of methods well known in the art. For example, the methods and assays of the invention may include, without limitation, the use of: RT-PCR, real-time RT-PCR, Oligonucleotide microarray or Northern blot.

In another aspect, there is provided a kit (or assay) comprising means for determining the level of UCP2 mRNA, and at least one of: i) means for isolating platelets from a sample and ii) instructions for determining the level of UCP2 mRNA in isolated platelets. The kit may optionally further comprise a control sample or data useful for comparing the level of UCP2 mRNA to its level in healthy control subject(s).

The following examples are presented in order to more fully illustrate some embodiments of the invention. They should, in no way be construed, however, as limiting the broad scope of the invention.

### EXAMPLES

### Example 1

### UCP2 and UCP3 Expression in Platelets

Platelet-Rich Plasma (PRP) Preparation and Platelet Purification

30 ml of EDTA-containing blood samples were left for 2 hours in room temperature and then centrifuged at 1200 RPM for 10 minutes, then the supernatant was removed and the procedure was repeated. The deposition (platelets) was centrifuged for another 15 minutes at 2500 RPM at 4° C., the supernatant was then removed and the platelets were re-suspended in 1 ml PBS (without calcium and magnesium). Platelet amount was determined by FACS (Beckman Coulter, LH500, Miami Fla.) and adjusted.

Platelet-UCP mRNA Isolation

RNA purification was performed using the EZ_RNA reagent (Biological Industry, Kibbutz Beit Haemek) according to the manufacturer's specifications. EZ-RNA is a complete kit with ready-to-use reagents for the isolation of total RNA from samples of human, animal, plant, yeast, bacterial and viral origin. It is based on disruption of cells in guanidine thiocyanate/detergent solution, followed by organic extraction and alcohol precipitation of the RNA, and allows simultaneous processing of a large number of samples. The resulting RNA is suitable for the isolation of Poly A+ RNA or for Northern Blotting, Dot Blotting, in vitro Translation, Molecular Cloning, RT-PCR and RNase Protection Assays, or other analytical procedures. mRNA content was determined by a Nanodrop ND-1000 instrument.

RT PCR was performed according to standard procedure. Real time PCR was performed according to standard procedure using primers for UCP2, UCP3 or beta actin (Sigma) as a control. The primers used were:

(SEQ ID NOs: 3-4 and 7-8, respectively)
UCP2-
5′-CCTCCTGAAAGCCAACCTCA-3′,
5′-AGAAGCCTGCCCCAAAGG-3;
UCP3-
5′-CACAGCCTTCTACAAGGGATTTACAC-3′,
5′-CTTCCATTCTTAACTGGTTTCGGA-3′.

Protein levels were determined in platelets by Western blot, as follows. UCP2 or UCP3 were immunopercipitated from 1 mg protein using anti-UCP2 or anti-UCP3 antibodies. The resulting protein was separated by 13% acrylamide gel, and transferred onto a nitrocellulose membrane. UCP2/3 protein was detected using the anti-UCP2 or anti-UCP3 antibodies used for immunoprecipitation and peroxidase-conjugated secondary antibodies, according to standard procedure.

The inventors now report that UCP2 and UCP3 are expressed in platelets. UCP2 and UCP3 mRNA and proteins were both identified in isolated platelets of healthy human subjects, while UCP1 was not identified to be expressed in the platelets.

### Example 2

### Effect of Medium Glucose Concentration and Incubation Time on UCP2 mRNA Levels

30 ml of blood was drawn from 7 healthy subjects and the platelets (3×10⁹/ml) were incubated in three wells with different glucose concentrations (100 mg/dL and 250 mg/dL for 24 hours, and 100 mg/dL with no incubation).

A significantly higher concentration of UCP2 mRNA was found with no incubation 0.055±0.02 compared to 0.019±0.011 and 0.011±0.007 (p<0.0027), for 24 hour incubation at 100 mg/dL and 250 mg/dL respectively). Thus, further experiments were performed on freshly isolated platelets without a preceding overnight incubation.

### Example 3

### UCP2 mRNA and Protein Levels in Platelets of Healthy, Non-Balanced and Balanced Diabetic Subjects

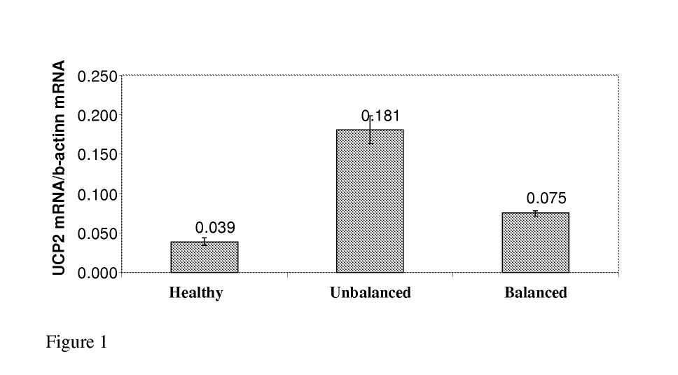

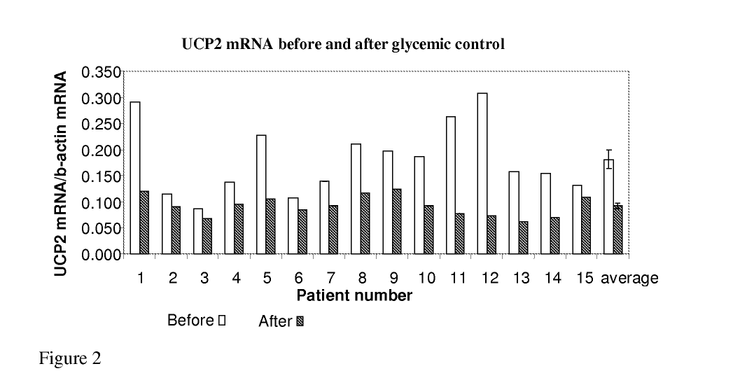

Blood was drawn from healthy subjects and newly diagnosed diabetic subjects as well as balanced diabetic patients (based on their HbA1c level). 1 microgram mRNA was tested using real time PCR as described in Example 1 above. The results in FIGS. 1 and 2 are presented as the ratio of UCP2 mRNA/beta actin mRNA (average±SD).

As can be seen in FIG. 1, UCP2 mRNA of non balanced subjects was significantly higher compared to healthy subjects 0.18±0.07 vs. 0.04±0.02 (p<0.0001) and balanced subjects 0.18±0.07 vs. 0.07±0.01 (p<0.0001). No significant differences were found between healthy and balanced type II diabetic patients (p=0.13).

Next, fifteen newly diagnosed or non-balanced type 2 diabetes mellitus (T2DM) subjects were tested before balance and after balance of their glycemic status (based on their HbA1c level). As can be seen in FIG. 2, UCP2 mRNA decreased in all subjects after achieving glycemic control (from 0.18±0.07 to 0.09±0.02 (p<0.0001).

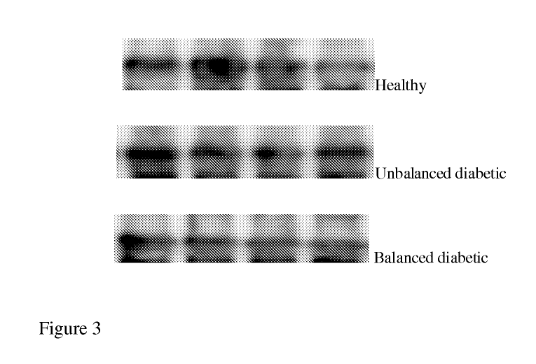

In contrast, no apparent changes were observed in UCP2 protein levels between the three tested groups (i.e. healthy subjects, newly diagnosed diabetic subjects and balanced diabetic patients, four subjects in each group, FIG. 3). In the healthy and balanced diabetic patient groups, the average UCP2 protein content was 169, with a standard deviation of 20.7 and 12.3, respectively. In the non-balanced diabetic group, the average UCP2 protein content was 177±11.8, p<0.35.

Similarly, no significant changes were observed in UCP2 protein levels in each subject before (176±11.8) or after balance (175±14.9), p<0.82.

### Example 4

### Levels of UCP2 mRNA and Metabolic Parameters Over Time

Platelets Isolation

Platelet-rich plasma (PRP) was obtained from whole blood collected in acid-citrate-dextrose solution B (ACD-B) in a volume ration of ACD-B to blood of 1:6 by centrifugation at 170×g for 10 minutes (min) at room temperature (RT). Anti coagulated blood was supplemented with 0.2 ug/ml PGE1 and 0.6 U/ml apyrase to reduce platelets' activation induced by in-vitro manipulations. The upper two parts of supernatant were then collected into Tyrode's Buffer (140 mM NaCl, 2.9 mM KCl, 0.36 mM NaH₂PO₄, pH 7.4, containing 5 mM dextrose, 0.2 ug/ml PGE1 and 0.6 U/ml apyrase). The PRP was then spun at 607×g for 10 minutes, 4° C. The pellet containing platelets was then re-suspended in 1 mL of Tyrode's-HEPES buffer (140 mM NaCl, 2.9 mM KCl, 0.36 mM NaH₂PO₄, 10 mM HEPES, pH 7.4, containing 5 mM dextrose) and Platelet concentration was determined using Beckman Coulter, LH500, FL, USA.

Platelet purity of the resulting preparation was >98%. A representative measure is presented below, in which the resulting platelets are isolated from other blood components:

White blood cells (WBC)—0.0 CL 10̂3/μL

Red blood cells (RBC)—0.0 L 10̂6/μL

Platelets (PLT)—100-300 10̂3/μL

Extraction of RNA and cDNA Preparation

Total RNA was extracted by EZ-RNA kit (Biological Industries Ltd., Israel) according to the manufacturer's instructions. The Concentration of mRNA was tested in Nanodrop ND1000 spectrophotometer (Thermo Scientific, Epson, Surrey, UK). One microgram of total RNA was reverse transcribed into cDNA using iScript cDNA Synthesis Kit (Bio-Rad Laboratories, Inc.).

Polymerase Chain Reaction

PCR based amplification of cDNA was carried out with UCP2 primers 5′-CCTCTGGATACTGCTAAAGT-3′ and 5′CCTTGATGAGGTCATAGGTC-3′ (SEQ ID NOs: 5-6, respectively). Respective PCR products were run on 1.5% agarose gel and viewed under UV Gel Doc using ethidium bromide staining

Real-Time PCR

cDNA obtained above was analyzed using quantitative real-time PCR to determine UCP2 expression. Real-time PCR was carried out in a 20 μl reaction volume using TaqMan assay (Hs01075227_ml) and Taqman gene expression Master mix (Life technologies, Carlsbad Calif., USA). To avoid amplification of genomic DNA, the primers were placed at the junction of two exons. Semi-quantitative real-time PCR was done using β-actin (Hs01060665_gl) as an internal control to normalize for gene expression.

Subjects and Study

Test subjects were unbalanced T2DM patients receiving insulin (subject 2) or insulin analogs (insulin glargine (Lantus), subject 1). Subjects were identified as having unbalanced glycemic control status based on HgbA1c and blood sugar level measurements. Prior to the test study, patient 1 (female) weighted 112 kg, with lean body mass (LBM) of 53.7 kg, 49.5% fat and body mass index (BMI) of 40.6. Patient 2 (male) weighted 105 kg, with LBM of 67.6, 33.8% fat and 36.8 BMI.

On day 0 of the study, the GLP-1 Receptor Agonist Vicroza® was added to the treatment course of both subjects, and they were instructed to undertake a low-calorie diet. Subjects were monitored for UCP2 mRNA and HgbA1c levels and for metabolic parameters: ATP production of platelets, oxygen consumption of platelets, FFA plasma concentration (free fatty acids or non-esterified fatty acids) and ROS plasma concentration every two weeks until week 6, then once a month. The predicted and actual resting metabolic rates (P. RMR and RMR, respectively) used to calculate the respiratory quotient (RQ, indicating the inherent composition and utilization of carbohydrates, fats and proteins as they are converted to energy) were also measured.

Results

Results for patients 1 and 2 are presented in Tables 1 and 2 below, respectively. At the beginning of the study, patients were determined to have an unfavorable metabolic status based on the readings of their metabolic parameters specified above. Both patients showed an overall improvement in glycemic and metabolic parameters over time from the onset of the new therapeutic regimen and diet. Caloric intake was limited in accordance with the prescribed diet, accompanied by body weight decrease. ATP production, cellular oxidation of platelets (O₂ intake) and RQ values generally increased over time, indicating an improvement in patients' metabolic status. Thus, the tested schedules were determined appropriate for both patients.

As can be seen in Table 1, in patient 1 UCP2 mRNA levels decreased over time, in correlation with improvement in other test parameters. Decrease in UCP2 was already detected at week 2 from the onset of treatment. HgA1c levels also decreased with treatment, however the decrease could only be detected as of week 18 of treatment. In patient 2 (Table 2), a similar decrease in UCP2 mRNA levels could be detected; parallel reduction in HgA1c levels could not be observed within the time frame of the experiment, i.e. up to week 18.

In Tables 1 and 2 below, UCP2 mRNA levels are presented in arbitrary units.

TABLE 1
Glycemic and metabolic parameters over time in patient 1

| | | Average | | | | | |
|---|---|---|---|---|---|---|---|
| | | Caloric | | | | | |
| | Weight | intake | | RMR | P. RMR | | HgbA1c |
| Week | (kg) | (kcal/day) | RQ | (kcal/day) | (kcal/day) | UCP2 mRNA | (%) |
| 0 | 112 | Received | 0.76 | 1883 | 1951 | 35.681 ± 0.133 | 12.1 |
| | | food diary | | | | | |
| 2 | 112.9 | 912 | — | — | — | 29.988 ± 0.053 | 12.1 |
| 4 | 112.9 | 1038 | — | — | — | 29.750 ± 0.100 | 12 |
| 6 | 110.4 | 1034 | — | — | — | 31.020 ± 0.019 | 12.1 |
| 10 | 111.3 | 995 | — | — | — | — | 12.2 |
| 14 | 110.6 | 911 | 0.76 | 1865 | 1975 | 30.429 ± 0.019 | 11.9 |
| 18 | 109.5 | — | — | — | — | — | 11.3 |
| 22 | 109.8 | 1125 | 0.79 | 1857 | 2017 | 28.960 ± 0.047 | 11.1 |

TABLE 2
Glycemic and metabolic parameters over time in patient 2

| Week | Weight (kg) | UCP2 mRNA | HgbA1c (%) |
|---|---|---|---|
| 0 | 105 | — | 7.7 |
| 2 | 102.9 | 33.858 ± 0.085 | 7.7 |
| 4 | 100 | — | 7.9 |
| 6 | 99.8 | 30.912 ± 0.131 | 7.8 |
| 10 | 98 | 27.300 ± 0.175 | 7.9 |
| 14 | 98 | — | 8 |
| 18 | 98.7 | — | 7.9 |

The results thus demonstrate that UCP2 mRNA levels may be used as a marker to monitor the alterations in glycemic as well as metabolic parameters over time during the course of treatment. This marker can therefore be used to assess treatment suitability to a subject and is advantageous over the use of HgA1c, since it can detect not only changes in glucose control (as does HgbA1c) but also metabolic changes, and can provide an input earlier than HgA1c and with improved clinical value.

### Example 5

### UCP2 Sequences

Primer and probe sequences may be readily prepared by the skilled artisan based on UCP2 sequences known in the art. For example, the following sequences for human UCP2 have been disclosed:
Human UCP2 mRNA—accession no.: NM_{—}003355 (CDS 381-1310); SEQ ID NO: 1:

cactgcgaag cccagctgcg cgcgccttgg gattgactgt
ccacgctcgc ccggctcgtc cgacgcgccc tccgccagcc
gacagacaca gccgcacgca ctgccgtgtt ctccctgcgg
ctcggacaca tagtatgacc attaggtgtt tcgtctccca
cccattttct atggaaaacc aaggggatcg ggccatgata
gccactggca gctttgaaga acgggacacc tttagagaag
cttgatcttg gaggcctcac cgtgagacct tacaaagccg
gattccggca gagttcctct atctcgtctt gttgctgatt
aaaggtgccc ctgtctccag tttttctcca tctcctggga
cgtagcagga aatcagcatc atggttgggt tcaaggccac
agatgtgccc cctactgcca ctgtgaagtt tcttggggct
ggcacagctg cctgcatcgc agatctcatc acctttcctc
tggatactgc taaagtccgg ttacagatcc aaggagaaag
tcaggggcca gtgcgcgcta cagccagcgc ccagtaccgc
ggtgtgatgg gcaccattct gaccatggtg cgtactgagg
gcccccgaag cctctacaat gggctggttg ccggcctgca
gcgccaaatg agctttgcct ctgtccgcat cggcctgtat
gattctgtca aacagttcta caccaagggc tctgagcatg
ccagcattgg gagccgcctc ctagcaggca gcaccacagg
tgccctggct gtggctgtgg cccagcccac ggatgtggta
aaggtccgat tccaagctca ggcccgggct ggaggtggtc
ggagatacca aagcaccgtc aatgcctaca agaccattgc
ccgagaggaa gggttccggg gcctctggaa agggacctct
cccaatgttg ctcgtaatgc cattgtcaac tgtgctgagc
tggtgaccta tgacctcatc aaggatgccc tcctgaaagc
caacctcatg acagatgacc tcccttgcca cttcacttct
gcctttgggg caggcttctg caccactgtc atcgcctccc
ctgtagacgt ggtcaagacg agatacatga actctgccct
gggccagtac agtagcgctg gccactgtgc ccttaccatg
ctccagaagg aggggccccg agccttctac aaagggttca
tgccctcctt tctccgcttg ggttcctgga acgtggtgat
gttcgtcacc tatgagcagc tgaaacgagc cctcatggct
gcctgcactt cccgagaggc tcccttctga gcctctcctg
ctgctgacct gatcacctct ggctttgtct ctagccgggc
catgctttcc ttttcttcct tctttctctt ccctccttcc
cttctctcct tccctctttc cccacctctt ccttccgctc
ctttacctac caccttccct ctttctacat tctcatctac
tcattgtctc agtgctggtg gagttgacat ttgacagtgt
gggaggcctc gtaccagcca ggatcccaag cgtcccgtcc
cttggaaagt tcagccagaa tcttcgtcct gcccccgaca
gcccagccta gcccacttgt catccataaa gcaagctcaa
ccttgg

Human UCP2 protein—accession no.: NP_{—}003346.2; SEQ ID NO: 2:

MVGFKATDVPPTATVKFLGAGTAACIADLITFPLDTAKVRLQIQGESQ
GPVRATASAQYRGVMGTILTMVRTEGPRSLYNGLVAGLQRQMSFASVR
IGLYDSVKQFYTKGSEHASIGSRLLAGSTTGALAVAVAQPTDVVKVRF
QAQARAGGGRRYQSTVNAYKTIAREEGFRGLWKGTSPNVARNAIVNCA
ELVTYDLIKDALLKANLMTDDLPCHFTSAFGAGFCTTVIASPVDVVKT
RYMNSALGQYSSAGHCALTMLQKEGPRAFYKGFMPSFLRLGSWNVVMF
VTYEQLKRALMAACTSREAPF

It is to be understood that such primer and probe sequences may be altered based on known or identified genetic variations in the UCP2 gene, as known in the art.

### REFERENCES

1. Amisten S. A Rapid and Efficient Platelet Purification Protocol for Platelet Gene Expression Studies. Methods Mol Biol. 2012;788:155-72.

2. Azzu V, Jastroch M, Divakaruni A S, Brand M D. The regulation and turnover of mitochondrial uncoupling proteins. Biochim Biophys Acta. 2010; 1797(6-7): 785-791.

3. Emre Y, Nubel T. Uncoupling protein UCP2: When mitochondrial activity meets immunity. FEBS Letters 584 (2010) 1437-1442.

4. Jia J J, Zhang X, Ge C R, Jois M. The polymorphisms of UCP2 and UCP3 genes associated with fat metabolism, obesity and diabetes. Obes Rev. 2009; 10(5):519-26.

5. Mailloux R J, Harper M E. Uncoupling proteins and the control of mitochondrial reactive oxygen species production. Free Radic Biol Med. 2011; 51(6):1106-15.

6. Marmontel de Souza B, Assmann T S, Klemann L M, Gross J L, Canani L H, Crispim D. The role of uncoupling protein 2 (UCP2) on the development of 2 diabetes mellitus and its chronic complications. Arq Bras Endocrinol Metabol. 2011; 55(4):239-48.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-7. (canceled)

8. A method for determining the metabolic status of a subject, comprising determining the level of Uncoupling Protein-2 (UCP2) mRNA specifically in platelets of said subject.

9. The method of claim 8 wherein the platelets are isolated from a blood sample obtained from the subject.

10. The method of claim 8, wherein a platelet UCP2 mRNA level that is significantly higher than its level in healthy control subjects indicates an unfavorable metabolic status in said subject.

11. The method of claim 8 wherein the subject is afflicted with a chronic disorder associated with elevated blood levels of glucose, reactive oxygen species (ROS) or free fatty acids (FFA).

12. The method of claim 8 wherein the disorder is selected from the group consisting of diabetes mellitus, a nutritional disorder, a chronic inflammatory disease and an endocrine disease.

13. The method of claim 12 wherein the disorder is selected from the group consisting of type II diabetes, type I diabetes, anorexia nervosa, obesity, syndrome X, sepsis, inflammatory bowel disease (IBD), rheumatoid arthritis (RA), chronic obstructive pulmonary disease (COPD), Hashimoto's thyroiditis and Graves' disease.

14. The method of claim 13, wherein said disorder is type II diabetes mellitus.

15. The method according to claim 8, wherein said method is used for determining the metabolic status of said subject within the preceding 5-14 days or within the preceding 5-7 days of platelet isolation.

16. The method of claim 14, wherein the method further comprises determining the glycemic control status of a subject, and wherein a platelet UCP2 mRNA level that is significantly higher than its level in healthy control subjects indicates an unfavorable glycemic control status in said subject.

17. The method according to claim 8, wherein the subject is receiving a treatment for the disorder, and wherein a platelet level of UCP2 mRNA that is significantly elevated compared to its level in healthy control subjects indicates that the treatment is unsuitable for said subject.

18. A method for evaluating a treatment for a disorder in a subject in need thereof, wherein the subject is receiving the treatment for the disorder, and wherein a platelet level of UCP2 mRNA that is significantly elevated compared to its level in healthy control subjects indicates that said treatment is unsuitable for said subject.

19. The method of claim 18, wherein said method is used for evaluating said treatment within 2-16 weeks from the onset of said treatment.

20. The method of claim 18 wherein the subject is afflicted with a chronic disorder associated with elevated blood levels of glucose, ROS or FFA selected from the group consisting of diabetes mellitus, a nutritional disorder, a chronic inflammatory disease and an endocrine disease.

21. The method of claim 20 wherein the disorder is selected from the group consisting of type II diabetes, type I diabetes, anorexia nervosa, obesity, syndrome X, sepsis, inflammatory bowel disease (IBD), rheumatoid arthritis (RA), chronic obstructive pulmonary disease (COPD), Hashimoto's thyroiditis and Graves' disease.

22. The method of claim 21, wherein said disorder is type II diabetes mellitus.

23. A kit comprising means for determining the level of Uncoupling Protein-2 (UCP2) mRNA, and at least one of: i) means for isolating platelets from a sample and ii) instructions for determining the level of UCP2 mRNA in isolated platelets.

24. The kit of claim 23, comprising instructions for determining the level of UCP2 mRNA in platelets isolated from a patient afflicted with type II diabetes, type I diabetes, anorexia nervosa, obesity, syndrome X, sepsis, inflammatory bowel disease (IBD), rheumatoid arthritis (RA), chronic obstructive pulmonary disease (COPD), Hashimoto's thyroiditis or Graves' disease.

25. The kit of claim 23, wherein the instructions indicate that a platelet UCP2 mRNA level that is significantly higher than its level in healthy control subjects indicates an unfavorable metabolic status in said subject.

26. The kit of claim 23, wherein the instructions indicate that a platelet UCP2 mRNA level that is significantly higher than its level in healthy control subjects indicates an unfavorable glycemic control status in said subject.

27. The kit of claim 23, wherein the determining the level of UCP2 mRNA is performed by a method selected from the group consisting of reverse-transcriptase polymerase chain reaction (RT-PCR), real-time RT-PCR, Oligonucleotide microarray and Northern blot.


SEQUENCE LISTING (8 sequences)

SEQ ID NO: 1 - DNA, 1646 bp, Homo sapiens
cactgcgaagcccagctgcgcgcgccttgggattgactgtccacgctcgcccggctcgtccgacgcgccctccgccagccgacagacacagccgcacgcactgccgtgttctccctgcggctcggacacatagtatgaccattaggtgtttcgtctcccacccattttctatggaaaaccaaggggatcgggccatgatagccactggcagctttgaagaacgggacacctttagagaagcttgatcttggaggcctcaccgtgagaccttacaaagccggattccggcagagttcctctatctcgtcttgttgctgattaaaggtgcccctgtctccagtttttctccatctcctgggacgtagcaggaaatcagcatcatggttgggttcaaggccacagatgtgccccctactgccactgtgaagtttcttggggctggcacagctgcctgcatcgcagatctcatcacctttcctctggatactgctaaagtccggttacagatccaaggagaaagtcaggggccagtgcgcgctacagccagcgcccagtaccgcggtgtgatgggcaccattctgaccatggtgcgtactgagggcccccgaagcctctacaatgggctggttgccggcctgcagcgccaaatgagctttgcctctgtccgcatcggcctgtatgattctgtcaaacagttctacaccaagggctctgagcatgccagcattgggagccgcctcctagcaggcagcaccacaggtgccctggctgtggctgtggcccagcccacggatgtggtaaaggtccgattccaagctcaggcccgggctggaggtggtcggagataccaaagcaccgtcaatgcctacaagaccattgcccgagaggaagggttccggggcctctggaaagggacctctcccaatgttgctcgtaatgccattgtcaactgtgctgagctggtgacctatgacctcatcaaggatgccctcctgaaagccaacctcatgacagatgacctcccttgccacttcacttctgcctttggggcaggcttctgcaccactgtcatcgcctcccctgtagacgtggtcaagacgagatacatgaactctgccctgggccagtacagtagcgctggccactgtgcccttaccatgctccagaaggaggggccccgagccttctacaaagggttcatgccctcctttctccgcttgggttcctggaacgtggtgatgttcgtcacctatgagcagctgaaacgagccctcatggctgcctgcacttcccgagaggctcccttctgagcctctcctgctgctgacctgatcacctctggctttgtctctagccgggccatgctttccttttcttccttctttctcttccctccttcccttctctccttccctctttccccacctcttccttccgctcctttacctaccaccttccctctttctacattctcatctactcattgtctcagtgctggtggagttgacatttgacagtgtgggaggcctcgtaccagccaggatcccaagcgtcccgtcccttggaaagttcagccagaatcttcgtcctgcccccgacagcccagcctagcccacttgtcatccataaagcaagctcaaccttgg

SEQ ID NO: 2 - protein, 309 aa, Homo sapiens
MVGFKATDVPPTATVKFLGAGTAACIADLITFPLDTAKVRLQIQGESQGPVRATASAQYRGVMGTILTMVRTEGPRSLYNGLVAGLQRQMSFASVRIGLYDSVKQFYTKGSEHASIGSRLLAGSTTGALAVAVAQPTDVVKVRFQAQARAGGGRRYQSTVNAYKTIAREEGFRGLWKGTSPNVARNAIVNCAELVTYDLIKDALLKANLMTDDLPCHFTSAFGAGFCTTVIASPVDVVKTRYMNSALGQYSSAGHCALTMLQKEGPRAFYKGFMPSFLRLGSWNVVMFVTYEQLKRALMAACTSREAPF

SEQ ID NO: 3 - DNA, 20 bp, Artificial Sequence
  PCR primer
cctcctgaaagccaacctca

SEQ ID NO: 4 - DNA, 18 bp, Artificial Sequence
  PCR primer
agaagcctgccccaaagg

SEQ ID NO: 5 - DNA, 20 bp, Artificial Sequence
  PCR primer
cctctggatactgctaaagt

SEQ ID NO: 6 - DNA, 20 bp, Artificial Sequence
  PCR primer
ccttgatgaggtcataggtc

SEQ ID NO: 7 - DNA, 26 bp, Artificial Sequence
  PCR primer
cacagccttctacaagggatttacac

SEQ ID NO: 8 - DNA, 24 bp, Artificial Sequence
  PCR primer
cttccattcttaactggtttcgga